(19) United States
(12) Patent Application Publication
(10) Pub. No.: US 2008/0221135 A1
(43) Pub. Date: Sep. 11, 2008

(54) COMBINATION THERAPY

(75) Inventor: Maurizio Voi, Stamford, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company, Princeton, NJ (US)

(21) Appl. No.: 11/382,737

(22) Filed: May 11, 2006

Related U.S. Application Data
(60) Provisional application No. 60/680,691, filed on May 13, 2005.

(51) Int. Cl. A61K 31/519; A61K 31/337; A61P 35/00

(52) U.S. Cl. 514/265.1; 514/449

(57) ABSTRACT

This invention relates to a combination of anti-cancer compounds which comprises a) an orally effective taxane, and b) a thymidylate synthase inhibitor, and optionally at least one pharmaceutically acceptable carrier for simultaneous, separate or sequential use.

### RELATED APPLICATION

This application claims priority benefit under Title 35 § 119(e) of U.S. Provisional Application No. 60/680,691, filed on May 13, 2005, incorporated herein by reference in its entirety.

### FIELD OF THE INVENTION

This invention relates to a combination of anti-cancer compounds which comprises a) an orally effective taxane, and b) a thymidylate synthase inhibitor, and optionally at least one pharmaceutically acceptable carrier for simultaneous, separate or sequential use.

### BACKGROUND OF THE INVENTION

It is now generally accepted that tubulin polymerization is one of the most effective targets for cancer chemotherapy as evidenced by the clinical success of Taxol® (paclitaxel) and Taxotere® (docetaxel). Taxol®, alone or in combination with other cytotoxic agents, is active in a broad range of cancers, including breast, ovarian and non-small cell lung cancer (NSCLC). Paclitaxel is a schedule dependent drug that benefits from prolonged tumor exposure times. Unfortunately, the oral bioavailability of the commercially available taxanes (paclitaxel and docetaxel) is very low (<1% in rats). As such, while increased efficacy might be expected from more frequent administration or prolonged infusions, intravenous administration to achieve this is relatively inconvenient and inhibits the evaluation of potentially more efficacious schedules of paclitaxel and/or docetaxel. Thus, an oral taxane would be ideal for investigating such protracted regimens.

3′-tert-butyl-3′-N-tert-butyloxycarbonyl-4-deacetyl-3′-dephenyl-3′-N-debenzoyl-4-O-methoxycarbonyl-paclitaxel, having the structure of formula I:

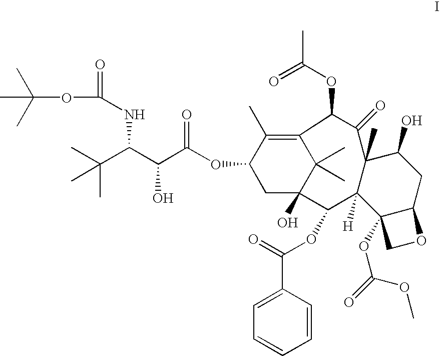

is an orally active C-4 methyl carbonate analog of paclitaxel that contains additional modifications to the side chain. This novel taxane has demonstrated preclinical anti-tumor activity comparable to intravenous (I.V.) paclitaxel. Compound I exhibits good oral bioavailability in both the rat and dog, and oral bioavailability of 24% (with a 45% coefficient of variation—CV) in humans.

Phase I data available to date suggests that Compound I shows anti-tumor activity when given on a continuous weekly schedule at doses of 120 mg/m² and higher. It has also been found that Compound I given on a twice weekly schedule at doses of 60 mg/m² and higher may reduce dose limiting toxicities seen on a weekly schedule. Tolerability is also acceptable at doses of 120 and 200 mg/m² on this schedule.

Pemetrexed (Alimta®) is a multi-targeted antifolate antineoplastic agent that exerts its action by disrupting folate-dependent metabolic processes essential for cell replication. Pemetrexed has shown anti-tumor activity in pre-clinical models against a variety of tumor types, including NSCLC. It has been shown to be relatively well tolerated when administered once every 21 days with appropriate vitamins supplementation with a low incidence of severe myelosuppression and with severe fatigue and transient transaminases elevation being the most frequently reported non-hematological toxicities. Phase II, and more recently, Phase III studies have confirmed a manageable toxicity profile and its activity, especially in the treatment of mesothelioma and in pre-treated NSCLC.

Pemetrexed has been combined with a variety of other chemotherapy agents, such as gemcitabine, docetaxel, paclitaxel, platinums, and vinorelbine in the treatment of NSCLC showing promising activity and good tolerability. Pemetrexed (Alimta®) was approved for the 2nd line treatment of NSCLC by the US FDA in August 2004. It has also been approved in Europe by the Committee for Proprietary Medicinal Products (CPMP) in September 2004.

The current Phase I/II study is designed to explore the safety and efficacy of escalating doses of Compound I and pemetrexed in patients with NSCLC who have received prior treatment for their advanced or metastatic disease.

Compound I is disclosed and claimed in U.S. Pat. No. 6,750,246 which describes C-4 methyl carbonate taxane analogs which have been shown to possess surprising oral activity and thus would have utility against proliferative diseases after oral administration. WO 03/053350 discloses pharmaceutical compositions of orally effective taxane derivatives and their use for inhibiting tumor growth in mammalian hosts. The entire disclosures of each of the aforementioned patents and patent publications are incorporated herein by reference.

A pending U.S. patent application disclosing particular crystalline forms of Compound I was filed Nov. 22, 2005 as U.S. Ser. No. 11/285,463.

Pemetrexed is described and claimed in U.S. Pat. No. 5,344,932. U.S. Pat. No. 6,579,877 discloses and claims the combination of antifolates to reduce the toxicities associated with the administration of non-competitive thymidylate synthase inhibitors, such as pemetrexed.

### SUMMARY OF THE INVENTION

This invention relates to a combination of anti-cancer compounds which comprises a) a taxane, and b) a thymidylate synthase inhibitor, and optionally at least one pharmaceutically acceptable carrier for simultaneous, separate or sequential use.

In particular, the invention relates to a combination of anti-cancer compounds which comprises a) 3′-tert-butyl-3′-N-tert-butyloxycarbonyl-4-deacetyl-3′-dephenyl-3′-N-debenzoyl-4-O-methoxycarbonyl-paclitaxel, and b) pemetrexed, given at a dose and schedule as described herein.

### DETAILED DESCRIPTION OF THE INVENTION

The nature of proliferative diseases like solid tumor diseases is multifactorial. Under certain circumstances, drugs with different mechanisms of action may be combined. However, just considering any combination of drugs having different modes of action does not necessarily lead to combinations with advantageous effects. In fact, drugs within the same class may not all have the same effect when used in combination.

Pemetrexed has shown single-agent activity in the treatment of chemo-naive or pre-treated NSCLC with an acceptable tolerability profile; Compound I has also shown promising early signs of activity in the treatment of pre-treated NSCLC with transient, manageable neuropathy being the main toxicity. The toxicity profiles of the two compounds do not seem to overlap.

While two-drug combination therapy has proven superior to single-agent therapy in the first-line setting in the treatment of NSCLC, no definitive Phase III trials have been reported yet in the second-line setting. Therefore, the purpose of this study is to determine the feasibility of combining pemetrexed and Compound I, assessing the toxicity profile of this combination and recommending doses to be evaluated in a Phase II setting. If the combination of pemetrexed and Compound I appears to have promising activity in patients with pre-treated NSCLC and good tolerability, further evaluation of this combination regimen in the treatment of pre-treated NSCLC may be warranted comparing it to single-agent pemetrexed and single-agent Compound I.

Compound I may form salts which are also within the scope of this invention. Pharmaceutically acceptable (i.e. non-toxic, physiologically acceptable) salts are preferred, although other salts are also useful, e.g., in isolating or purifying the compounds of this invention.

Compound I may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The compounds for formula I may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art.

In addition, zwitterions (“inner salts”) may be formed.

All stereoisomers of the compounds of the instant invention are contemplated, either in admixture or in pure or substantially pure form. The definition of compounds according to the invention includes all the possible stereoisomers and their mixtures. Particularly preferred are the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

The combination of the invention may be useful in the treatment of a variety of cancers, including (but not limited to) the following:
carcinoma, including that of the bladder, breast, colon, kidney, liver, lung, including small cell lung cancer, esophagus, gall bladder, ovary, pancreas, stomach, cervix, thyroid, prostate, and skin, including squamous cell carcinoma;
hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkitt's lymphoma;
hematopoietic tumors of myeloid lineage, including acute and chronic myelogenous leukemias, myelodysplastic syndrome and promyelocytic leukemia;
tumors of mesenchymal origin, including fibrosarcoma and rhabdomyosarcoma;
tumors of the central and peripheral nervous system, including astrocytoma, neuroblastomal glioma and schwannomas; and
other tumors, including melanoma, seminoma, teratocarcinoma, osteosarcoma, xenoderoma pigmentosum, keratoctanthoma, thyroid follicular cancer and Kaposi's sarcoma.

### EXAMPLES

### Study Design

This is an open-label, Phase I/II multi-center study in patients with pre-treated NSCLC. Patients must have received one prior treatment with a cisplatin- or carboplatin-based regimen. In the Phase I portion of the study, they will receive escalating doses of pemetrexed administered I.V. over an approximately 10 minute infusion at a starting dose of 400 mg/m² repeated once every 21 days (1 cycle), concomitantly with escalating doses of Compound I administered orally (P.O.) on a schedule yet to be determined of each 21-Day cycle. Patients must take at least 5 daily doses of folic acid during the 7-day period preceding the first dose of pemetrexed. Dosing with folic acid should continue during the full course of treatment with pemetrexed and for 21 days after the last dose of pemetrexed. Patients must also receive vitamin B12 1000 μg via intramuscular injection beginning approximately 1 week prior to the first dose of pemetrexed and every three cycles thereafter.

These subsequent injections can be given on the same day as the dose of pemetrexed. Dexamethasone 4 mg orally twice per day will be taken on the day before, the day of, and the day after each dose of pemetrexed, unless contraindications exist.

Compound I will be given P.O. at the MTD on a schedule yet to be determined. It is anticipated that Compound I will be administered on a twice weekly schedule starting on Cycle 1, on Day 1. On Cycle 2, Day 1, Compound I will not be administered while pemetrexed infusion will be administered.

In the Phase II portion of the study, up to 40 patients will receive the combination of Compound I and pemetrexed at the doses recommended by the Phase I portion of the study. Supplementation with folic acid, vitamin B12 and dexamethasone will continue as received in the Phase I portion of this study. A one-stage design will be used to examine the tumor response rate of the combination of Compound I and pemetrexed. Patients will be assessed at regular intervals during treatment. Patients' lung cancer symptoms will be assessed at regular intervals. It is anticipated that approximately 10 study centers will participate, and that 12 months will be required to complete accrual.

### Materials and Methods

Test Product, Dose and Mode of Administration, Duration of Treatment: Compound I will be supplied by the Sponsor. Compound I will be supplied as 25-mg capsules and 5-mg capsules. Compound I is solubilized in PEG 400/PEG 1450/Gelucire 44/14 at a loading of 4% w/w, and filled into size #0, (25-mg) and size #1 (5-mg) gray opaque hard gelatin capsules. Pemetrexed will be supplied or reimbursed by the Sponsor in sterile, single-use vials containing 500 mg of pemetrexed. Folic acid, vitamin B12 and dexamethasone are commercially available. The initial starting dose of pemetrexed will be 400 mg/m² administered I.V. on Day 1 of a 21-day cycle over an approximately 10 minute infusion concomitantly with the administration of Compound I. Compound I will be administered at a starting dose to be determined. It is anticipated that the dose will be 60 mg/m² given PO as a single dose twice a week beginning on Day 1 of each 21-day cycle on a twice weekly schedule. Patients must take at least 5 daily doses of folic acid during the 7-day period preceding the first dose of pemetrexed. Dosing with folic acid should continue during the full course of treatment with pemetrexed and for 21 days after the last dose of pemetrexed. Patients must also receive vitamin B 12 1000 μg via intramuscular injection beginning approximately 1 week prior to the first dose of pemetrexed and every three cycles thereafter. These subsequent injections can be given on the same day as the dose of pemetrexed. Dexamethasone (4 mg orally twice per day) will be taken on the day before, the day of, and the day after each dose of pemetrexed, unless contraindications exist. Treatment duration will consist of at least 2 cycles (a cycle consisting of 6 twice weekly administrations of PO Compound I given twice a week for 3 consecutive weeks and of I.V. pemetrexed given once every 21 days). Duration of treatment will be based on tumor reassessments performed every 6 weeks. Patients with progressive disease (PD) will discontinue treatment with both study drugs. Patients with stable disease (SD) or a partial response (PR) will receive treatment until PD or for as long as it is in the patient's best interest to continue treatment, up to a maximum of 18 cycles. Patients with SD or PR may receive additional treatment with one or both study drugs beyond the 18th cycle if it is in their best interest to do so, and is agreed to by both the Investigator and Sponsor. Patients who achieve a complete response (CR) will receive treatment for up to a maximum of 4 cycles after confirmation of CR. Patients who omit treatment with Compound I or pemetrexed because of unacceptable toxicity may continue treatment with pemetrexed or Compound I alone at the discretion of the treating physician until unacceptable toxicity or PD.

Criteria for Evaluation: Data on toxicity occurring during the first cycle of treatment will be evaluated for the purpose of DLT and MTD determination on all patients enrolled in the Phase I portion of the study who received at least 1 dose of Compound I and/or pemetrexed. Adverse events and other symptoms will be graded according to the NCI CTCAE Version 3.0. The primary efficacy outcome of the Phase II portion of the study is response rate evaluated according to the modified WHO Criteria among response-evaluable patients. The study design requires at least 40 response-evaluable patients to be treated at the recommended Phase II doses of the combination of Compound I and pemetrexed. To be considered response-evaluable, patients must have been diagnosed with measurable NSCLC, received at least one recommended Phase II dose of Compound I or pemetrexed, and, for responders only, had a tumor reassessment performed. PK parameters (Cmax, Tmax, AUC (INF), and T-HALF) will be derived from Compound I plasma concentration versus time data. Cmax, AUC (INF), Vss, CLT, and T-HALF of pemetrexed will be derived from plasma concentration versus time data. Safety data will be analyzed on all patients treated in each dose level in both the Phase I and the Phase portions of the study, and who have received at least 1 dose of Compound I and/or pemetrexed. Lung cancer symptoms will be assessed using the 7-item disease specific Lung Cancer Symptom subscale of the Functional Assessment of Cancer Therapy-Lung (FACT-L).

Parenteral administration of drugs is not conducive to protracted, repetitive, chronic treatment applications. The recent advent of weekly regimens for the delivery of taxanes clinically, and the apparent success associated with their deployment in this manner, provides an impetus for the development of oral versions with at least comparable efficacy and no worse a toxicological profile. With the availability of a clinically active oral taxane, a wide range of different schedule options becomes feasible.

Despite advances in the past decade, patients with NSCLC and other tumors are in need of more effective therapeutic interventions. It is hoped that the combination of the invention will provide another option in treating NSCLC and other cancers.

We claim:

1. A pharmaceutical combination of anti-cancer compounds which comprises
a) a taxane, and
b) a thymidylate synthase inhibitor,
in which the active ingredients are present in each case in free form or as a pharmaceutically acceptable salt, solvate or ester.

2. The combination according to claim 1 wherein the taxane is a compound of the formula 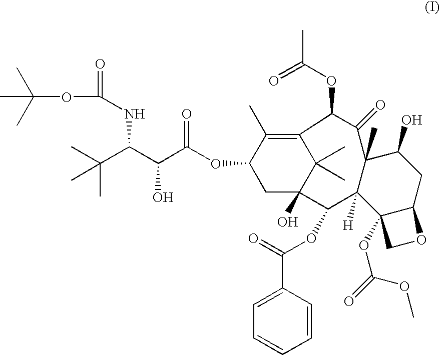
or a pharmaceutically acceptable salts, solvate, ester or isomer thereof,
and the thymidylate synthase inhibitor is pemetrexed.

3. The combination according to claim 2 wherein the taxane is administered orally.

4. The combination according to claim 2 wherein the thymidylate synthase inhibitor is administered intravenously.

5. The combination according to claim 4 wherein the thymidylate synthase inhibitor is administered at a starting dose of 400 mg/m² repeated once every 21 days.

6. The combination according to claim 5 wherein the taxane is administered on a twice weekly schedule at a starting dose of 60 mg/m².

7. The combination according to claim 6 wherein the anti-cancer agents are administered on a 21 day cycle.

8. A method for the treatment of cancer which comprises administering a therapeutically effective amount of
a) a taxane, and
b) a thymidylate synthase inhibitor,
in which the active ingredients are present in each case in free form or as a pharmaceutically acceptable salt, solvate or ester.

9. The method of claim 8 wherein the taxane is a compound of the formula 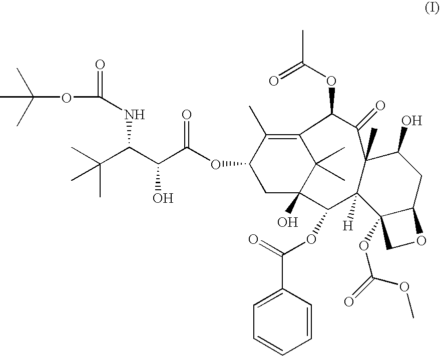
or a pharmaceutically acceptable salts, solvate, ester or isomer thereof.

10. The method according to claim 9 wherein the thymidylate synthase inhibitor is pemetrexed.

11. The method according to claim 10 wherein the taxane is administered orally at a therapeutically effective dose of 60 mg/m2 to 100 mg/m2 on a twice weekly dosing schedule.

12. The method according to claim 11 wherein pemetrexed inhibitor is administered intravenously at a therapeutically effective starting dose of 400 mg/m² repeated once every 21 days.

13. The method according to claim 12 wherein the cancer being treated is selected from colorectal cancer, breast cancer, gastric cancer, ovarian cancer, non-small cell lung cancer and cancers of the head and neck.

14. The method according to claim 13 wherein the cancer being treated is non-small cell lung cancer.
